(19) United States
(12) Patent Application Publication
(10) Pub. No.: US 2022/0284986 A1
(43) Pub. Date: Sep. 8, 2022

(54) SYSTEMS AND METHODS FOR IDENTIFYING EXON JUNCTIONS FROM SINGLE READS

(71) Applicant: Life Technologies Corporation, Carlsbad, CA (US)

(72) Inventors: Paolo Vatta, San Mateo, CA (US); Onur Sakarya, Redwood City, CA (US); Heinz Breu, Palo Alto, CA (US); Liviu Popescu, Sunnyvale, CA (US); Asim Siddiqui, San Francisco, CA (US); Fiona Hyland, San Mateo, CA (US)

(73) Assignee: Life Technologies Corporation, Carlsbad, CA (US)

(21) Appl. No.: 17/699,439

(22) Filed: Mar. 21, 2022

Related U.S. Application Data
(63) Continuation of application No. 15/928,202, filed on Mar. 22, 2018.
Application No. 15/928,202 is a continuation of application No. 13/097,328, filed on Apr. 29, 2011, now abandoned.
(60) Provisional application No. 61/426,826, filed on Dec. 23, 2010.
(60) Provisional application No. 61/330,118, filed on Apr. 30, 2010.

(51) Int. Cl. G16B 30/10; G16B 30/00

(52) U.S. Cl. CPC G16B 30/10; G16B 30/00

(57) ABSTRACT

Identification of exon junctions includes obtaining a first read sequence based on a detected plurality of signals of a first sequence. A list of exon prefix and suffix sequences are generated by identifying exons of the human genome with a prefix sequence mapping to a suffix sequence of the first read sequence and by identifying exons with a suffix sequence mapping to a prefix sequence of the first read sequence. A pair of exon sequences is selected, with a first exon sequence being one of the exon suffix sequences and a second exon sequence being one of the exon prefix sequences. Summing a number of sequence elements of the first exon sequence that overlap the prefix of the first read sequence, a number of sequence elements of the second exon sequence that overlap the suffix of the first read sequence, and a constant is used to identify a fusion junction.

### CROSS REFERENCE TO RELATED APPLICATIONS

This application is a continuation of U.S. application Ser. No. 15/928,202, filed Mar. 22, 2018, which is a continuation of U.S. application Ser. No. 13/097,328 filed Apr. 29, 2011 (now abandoned), which claims priority to U.S. Application 61/426,826 filed Dec. 23, 2010 (now expired) and U.S. Application No. 61/330,118 filed Apr. 30, 2010 (now expired), each of which disclosures are herein incorporated by reference in their entirety.

### FIELD

The present disclosure relates to biomolecule sequencing and in particular to systems and methods for identifying exon junctions.

### INTRODUCTION

Nucleic acid sequence information can be an important data set for medical and academic research endeavors. Sequence information can facilitate medical studies of active disease and genetic disease predispositions, and can assist in rational design of drugs (e.g., targeting specific diseases, avoiding unwanted side effects, improving potency, and the like). Sequence information can also be a basis for genomic and evolutionary studies and many genetic engineering applications. Reliable sequence information can be critical for other uses of sequence data, such as paternity tests, criminal investigations and forensic studies.

Sequencing technologies and systems, such as, for example, those provided by Applied Biosystems/Life Technologies (SOLiD Sequencing System), Solexa (Illumina), and 454 Life Sciences (Roche) can provide high throughput DNA/RNA sequencing capabilities to the masses. Applications which may benefit from these sequencing technologies include, but are certainly not limited to, targeted resequencing, miRNA analysis, DNA methylation analysis, whole-transcriptome analysis, and cancer genomics research.

Sequencing platforms can vary from one another in their mode of operation (e.g., sequencing by synthesis, sequencing by ligation, pyrosequencing, etc.) and the type/form of raw sequencing data that they generate. Generally, however, sequencing systems incorporating NGS technologies can produce a large number of short reads. As a result, these sequencing systems must be able to map a large number of reads against a genome in a relatively short amount of time. For a human size genome, for example, a sequencing system must map billions of reads.

A genome is a set of chromosomes, each chromosome is a double-stranded fragment of deoxyribonucleic acid (DNA), and each strand is a sequence of bases; A, C, G, and T, for example. A gene is a subsequence of a strand, and an exon is a subsequence of a gene. The biological process of transcription creates a single-stranded ribonucleic acid (RNA) transcript. An exon-exon junction, or simply junction when there is no ambiguity, is two adjacent exons on a transcript. Normally, a transcript is made up of exons transcribed from a single gene, and a single gene may have more than one transcript. Additionally, fusion junctions include the two exons from different genes, perhaps even from different chromosomes.

### SUMMARY

In various embodiments, an exon junction can be identified from a read of a transcript spanning the exon junction. The exon junction can include two adjacent exons in a transcript. The exons can come from a single gene or be a product of a gene fusion between two different genes. A prefix of the read can be mapped to a first exon and a suffix of the read can be mapped to a second exon. A junction can be identified when the number of sequence elements in the read sequence substantially equals a sum of a number of sequence elements of the exons that overlap portions of the read sequence and a constant.

### BRIEF DESCRIPTION OF THE DRAWINGS

The skilled artisan will understand that the drawings, described below, are for illustration purposes only. The drawings are not intended to limit the scope of the present teachings in any way.

FIG. 1 is a diagram showing suffixes of exons that map to the prefix of a read sequence, in accordance with various embodiments.

FIG. 2 is a diagram showing prefixes of exons that map to the suffix of a read sequence, in accordance with various embodiments.

FIG. 3 is a diagram showing a pair of exons that map to a read sequence and identify an exon junction, in accordance with various embodiments.

FIG. 4 is an exemplary flowchart showing a method for identifying an exon junction from a single read of a transcript, in accordance with various embodiments.

FIG. 5 is an exemplary flow diagram showing an additional method for identifying an exon junction, in accordance with various embodiments.

FIG. 6 is a block diagram that illustrates a computer system, in accordance with various embodiments.

FIG. 7 is a schematic diagram of a system of distinct software modules that performs a method for identifying an exon junction from a single read of a transcript, in accordance with various embodiments.

FIG. 8 is schematic diagram of a system for identifying an exon junction from a single read of a transcript, in accordance with various embodiments.

It is to be understood that the figures are not necessarily drawn to scale, nor are the objects in the figures necessarily drawn to scale in relationship to one another. The figures are depictions that are intended to bring clarity and understanding to various embodiments of apparatuses, systems, and methods disclosed herein. Wherever possible, the same reference numbers will be used throughout the drawings to refer to the same or like parts.

### DESCRIPTION OF VARIOUS EMBODIMENTS

The section headings used herein are for organizational purposes only and are not to be construed as limiting the described subject matter in any way. All literature and similar materials cited in this application, including but not limited to, patents, patent applications, articles, books, treatises, and internet web pages are expressly incorporated by reference in their entirety for any purpose. When definitions of terms in incorporated references appear to differ from the definitions provided in the present teachings, the definition provided in the present teachings shall control. It will be appreciated that there is an implied “about” prior to the temperatures, concentrations, times, etc. discussed in the present teachings, such that slight and insubstantial deviations are within the scope of the present teachings. In this application, the use of the singular includes the plural unless specifically stated otherwise. Also, the use of “comprise”, “comprises”, “comprising”, “contain”, “contains”, “containing”, “include”, “includes”, and “including” are not intended to be limiting. It is to be understood that both the foregoing general description and the following detailed description are exemplary and explanatory only and are not restrictive of the present teachings.

Unless otherwise defined, scientific and technical terms used in connection with the present teachings described herein shall have the meanings that are commonly understood by those of ordinary skill in the art. Further, unless otherwise required by context, singular terms shall include pluralities and plural terms shall include the singular. Generally, nomenclatures utilized in connection with, and techniques of, cell and tissue culture, molecular biology, and protein and oligo- or polynucleotide chemistry and hybridization described herein are those well-known and commonly used in the art. Standard techniques are used, for example, for nucleic acid purification and preparation, chemical analysis, recombinant nucleic acid, and oligonucleotide synthesis. Enzymatic reactions and purification techniques are performed according to manufacturer's specifications or as commonly accomplished in the art or as described herein. The techniques and procedures described herein are generally performed according to conventional methods well known in the art and as described in various general and more specific references that are cited and discussed throughout the instant specification. See, e.g., Sambrook et al., Molecular Cloning: A Laboratory Manual (Third ed., Cold Spring Harbor Laboratory Press, Cold Spring Harbor, N.Y. 2000). The nomenclatures utilized in connection with, and the laboratory procedures and techniques described herein are those well-known and commonly used in the art.

As utilized in accordance with the embodiments provided herein, the following terms, unless otherwise indicated, shall be understood to have the following meanings:

As used herein, “a” or “an” means “at least one” or “one or more”. Further, unless expressly stated to the contrary, “or” refers to an inclusive-or and not to an exclusive-or. For example, a condition A or B is satisfied by any one of the following: A is true (or present) and B is false (or not present), A is false (or not present) and B is true (or present), and both A and B are true (or present).

The phrase “next generation sequencing” refers to sequencing technologies having increased throughput as compared to traditional Sanger- and capillary electrophoresis-based approaches, for example with the ability to generate hundreds of thousands of relatively small sequence reads at a time. Some examples of next generation sequencing techniques include, but are not limited to, sequencing by synthesis, sequencing by ligation, and sequencing by hybridization. More specifically, the SOLiD Sequencing System of Life Technologies Corp. provides massively parallel sequencing with enhanced accuracy. The SOLiD System and associated workflows, protocols, chemistries, etc. are described in more detail in PCT Publication No. WO 2006/084132, entitled “Reagents, Methods, and Libraries for Bead-Based Sequencing,” international filing date Feb. 1, 2006, U.S. patent application Ser. No. 12/873,190, entitled “Low-Volume Sequencing System and Method of Use,” filed on Aug. 31, 2010, and U.S. patent application Ser. No. 12/873,132, entitled “Fast-Indexing Filter Wheel and Method of Use,” filed on Aug. 31, 2010, the entirety of each of these applications being incorporated herein by reference thereto.

The phrase “sequencing run” refers to any step or portion of a sequencing experiment performed to determine some information relating to at least one biomolecule (e.g., nucleic acid molecule).

The phrase “ligation cycle” refers to a step in a sequence-by-ligation process where a probe sequence is ligated to a primer or another probe sequence.

The phrase “color call” refers to an observed dye color that results from the detection of a probe sequence after a ligation cycle of a sequencing run. Similarly, other “calls” refer to the distinguishable feature observed.

The phrase “fragment library” refers to a collection of nucleic acid fragments, wherein one or more fragments are used as a sequencing template. A fragment library can be generated, for example, by cutting or shearing a larger nucleic acid into smaller fragments. Fragment libraries can be generated from naturally occurring nucleic acids, such as bacterial nucleic acids. Libraries comprising similarly sized synthetic nucleic acid sequences can also be generated to create a synthetic fragment library.

The phrase “mate-pair library” refers to a collection of nucleic acid sequences comprising two fragments having a relationship, such as by being separated by a known number of nucleotides. Mate pair fragments can be generated by cutting or shearing, or they can be generated by circularizing fragments of nucleic acids with an internal adapter construct and then removing the middle portion of the nucleic acid fragment to create a linear strand of nucleic acid comprising the internal adapter with the sequences from the ends of the nucleic acid fragment attached to either end of the internal adapter. Like fragment libraries, mate-pair libraries can be generated from naturally occurring nucleic acid sequences. Synthetic mate-pair libraries can also be generated by attaching synthetic nucleic acid sequences to either end of an internal adapter sequence.

The term “template” and variations thereof refer to a nucleic acid sequence that is a target of nucleic acid sequencing. A template sequence can be attached to a solid support, such as a bead, a microparticle, a flow cell, or other surface or object. A template sequence can comprise a synthetic nucleic acid sequence. A template sequence also can include an unknown nucleic acid sequence from a sample of interest and/or a known nucleic acid sequence.

The phrase “template density” refers to the number of template sequences attached to each individual solid support.

In various embodiments, a junction finding method can be used to find exon junctions. Junctions can be found using as input a set of small reads, obtained by sequencing a portion of a transcript, and a list of the exons within a genome. The reads can have a length of at least about 25 bases. Further, the length of the read can be not greater than about 10,000 bases, such as not greater than about 5000 bases, such as not greater than about 2000 bases, even not greater than about 1000 bases. For example, the read length can be not greater than about 750 bases, such as not greater than about 500 bases, such as not greater than about 250 bases, such as not greater than about 100 bases, such as not greater than about 75 bases, even not greater than about 50 bases. In particular embodiments, the length of the read can be short enough to span only a single exon junction. In other embodiments, the read can span one or more entire exons and additional portions from two exons flanking either side of the exon. The algorithm considers a read to be evidence of a junction between exon e and exon f, if the sequence of the read is a substring of the transcript that spans the junction site. Note that this definition is asymmetric; evidence for a junction between e and f is not evidence of a junction between and f and e.

An exon junction is where two adjacent exons on a transcript meet. The two adjacent exons can come from the same gene, from different genes, or even from different chromosomes. Of particular significance are gene fusions which are exon junctions spanning exons from two different genes. Gene fusions can arise from mutations including translocations, deletions, inversions, or trans-splicing. Gene fusions are thought to cause tumorigenesis by over activating proto-oncogenes, deactivating tumor suppressors, or altering the regulation or splicing of other genes which lead to defects in key signaling pathways.

In various embodiments, evidence of junctions can be provided by mapping reads to a fused exon pair. For example, a single read, either from a fragment library or a mate-pair library, can be identified to span the fused exon pair. In another example, a pair of reads can be identified from a mate-pair that spans the exon junction, with one read mapping to a first exon and the other read mapping to a second exon. Analysis of both single reads and mate-pairs that span an exon-exon junction can provide an increased confidence that an exon-exon junction exists within a transcriptome.

Junction candidates could be generated by testing all ordered pairs of exons against all reads. Each individual test could entail mapping the read to the fused exon pair to determine if it spans the junction point. All of this might take some time. For example, a file of 200 thousand exons and a file of 60 million reads would generate (2×10⁵)×(2×10⁵)×(6×10⁷)=2.4×10¹⁸ tests. If a million tests were executed each second, it would take about 76 thousand years to complete all the tests.

In various embodiments, a junction finding method can be used to search all exons for each read, rather than testing all reads for each exon pair. A list of two more exons can be obtained. Each exon in the list can include its sequence and the reverse of that sequence. The suffix of each exon from the list can then be compared to the prefix of the read sequence. Either the sequence of an exon or the reverse of that sequence can be used. Each exon from the list that maps to the prefix of the read sequence can be added to a left set of exons.

In various embodiments, a list of sequences can be generated from the list of exons. The list of sequences can include sequences from the suffix of each exon that have lengths between a minimum and maximum match length. For example, for a read sequence having a length of 50 and using a minimum match length of 10, the maximum match length can be 40 since 10 nucleotides are required to match an exon on the other end of the read sequence. The list of sequences can include all sequences from the suffix of an exon having a length between 10 and 40. Additionally, the list of sequences can include sequences of length 10 to 40 from the suffix of the reverse of the exon.

In various embodiments, the list of sequences can be sorted based on sequence. When mapping the sequence read to the list of sequences, an efficient search, such as a binary search, of the list of sequences can be made to locate sequences that match the sequence read. Once a subset of sequences from the list has been identified, each sequence having a minimum match length can be compared to the sequence read to determine if the sequence matches the exon over the length of the sequence. In particular embodiments, an approximate string matching algorithm can be used to compare the exon sequence to the read sequence, thereby allowing for a small number of mismatches between the exon sequence and the read sequence.

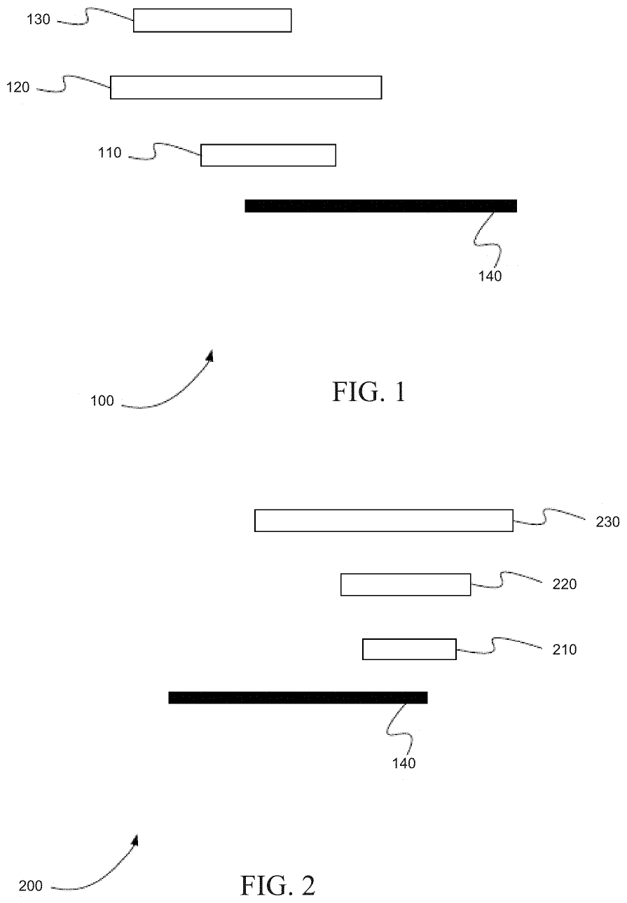

FIG. 1 is a diagram 100 showing suffixes of exons that map to the prefix of a read sequence 140, in accordance with various embodiments. The suffixes of the sequences of exons 110, 120, and 130 can overlap with or map to read sequence 140. Either the sequence of an exon or the reverse of that sequence can be used. Exons 110, 120, and 130, for example, can be added to the left set of exons.

Similarly, the prefix of each exon from the list can be compared to the suffix of the read sequence. Either the sequence of an exon or the reverse of that sequence can be used. Each exon from the list that maps to the suffix of the read sequence is added to a right set of exons.

FIG. 2 is a diagram 200 showing prefixes of exons that map to the suffix of a read sequence 240, in accordance with various embodiments. The prefixes of the sequences of exons 210, 220, and 230 overlap with or map to read sequence 240. Exons 210, 220, and 230, for example, are added to the right set of exons.

The number of sequence elements of each exon in the left set of exons that overlap with the read sequence can be added to the number of sequence elements of each exon in the right set of exons that overlap with the read sequence. In particular embodiments, the number of sequence elements of one or more exons that are mapped to a middle portion of the read sequence can be added to the sum of the number of sequence elements from the left and right exons. The total number of sequence elements of the two or more exon sequences that overlap can be compared to the length of the read sequence. If the exon sequences are mapped to the read sequence in base-space and the total number of sequence elements of the two or more exon sequences that overlap is equal to the length of the read sequence, then the read identifies one or more exon junctions. If the exon sequences are mapped to the read sequence in a monobase color-space and the total number of sequence elements of the two or more exon sequences that overlap is equal to the length of the read sequence, then the read identifies one or more exon junctions. In a monobase color-space each base is encoded with a single color call, for example. If the exon sequences are mapped to the read sequence in a dibase color-space and the total number of sequence elements of two or more exons_that overlap is equal to the length of the read sequence plus one, then the read identifies one or more exon junctions. In a dibase color-space two bases are encoded with a single color call, for example. One of skill in the art would recognize that additional coding schemes where the symbol matches three or more bases can be used with a corresponding change in the constant that is added to the total length of the left and right exons. For example, for a symbol matching three bases, a constant of two can be used.

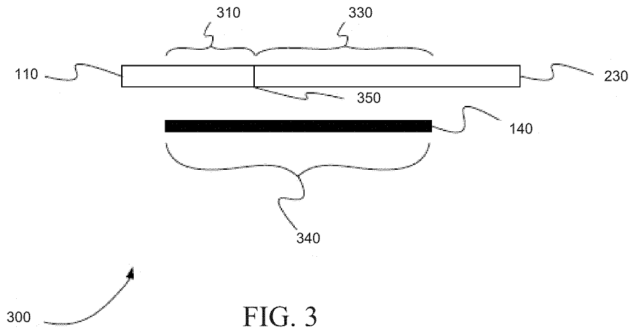

FIG. 3 is a diagram 300 showing a pair of exons that map to a read sequence 140 and identify an exon junction 350, in accordance with various embodiments. Exon 110 can map to the prefix of read sequence 140, and exon 230 can map to the suffix of read sequence 140. Overlap 310 can be the overlap of exon 110 with read sequence 140. Overlap 330 can be the overlap of exon 230 with read sequence 140. Because the sum of overlap 310 and overlap 330 is equal to the length 340 of read sequence 140, read sequence 140 can identify exon junction 350 of exon 110 and exon 230. This assumes, for example, that all sequences are base-space or mono-base sequences.

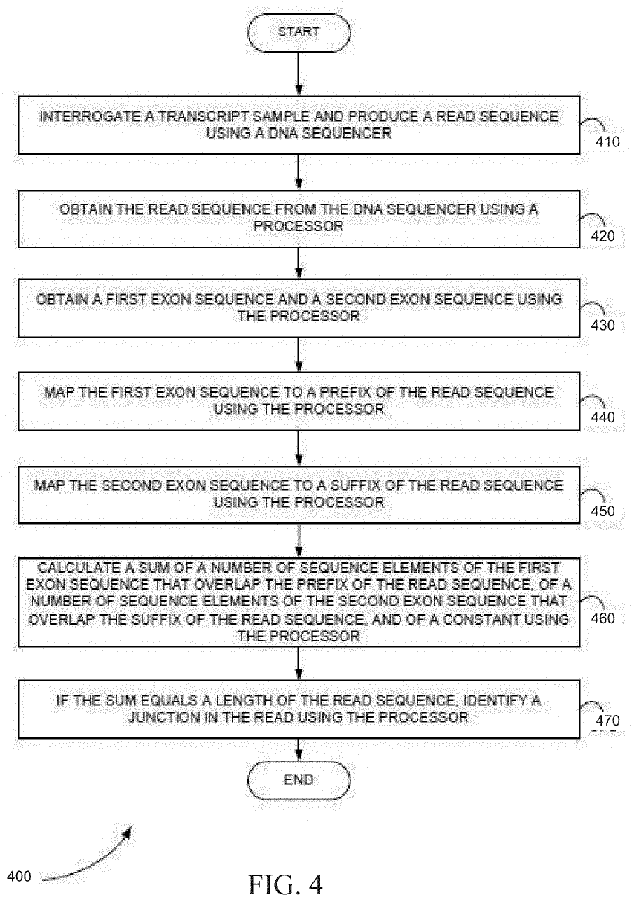

FIG. 4 is an exemplary flowchart showing a method 400 for identifying an exon junction from a single read of a transcript, in accordance with various embodiments.

At 410, a transcript sample can be interrogated and a read sequence can be produced using a nucleic acid sequencer.

At 420, the read sequence can be obtained from the nucleic acid sequencer using a processor.

At 430, a first exon sequence and a second exon sequence can be obtained using the processor.

At 440, the first exon sequence can be mapped to a prefix of the read sequence using the processor.

At 450, the second exon sequence can be mapped to a suffix of the read sequence using the processor.

At 460, a sum of a number of sequence elements of the first exon sequence that overlap the prefix of the read sequence, of a number of sequence elements of the second exon sequence that overlap the suffix of the read sequence, and of a constant can be calculated using the processor. In particular embodiments, the constant can depend of the encoding scheme, such as a monobase encoding scheme, a dibase encoding scheme, a tribase encoding scheme, and the like.

At 470, if the sum equals a length of the read sequence, a junction can be identified in the read using the processor.

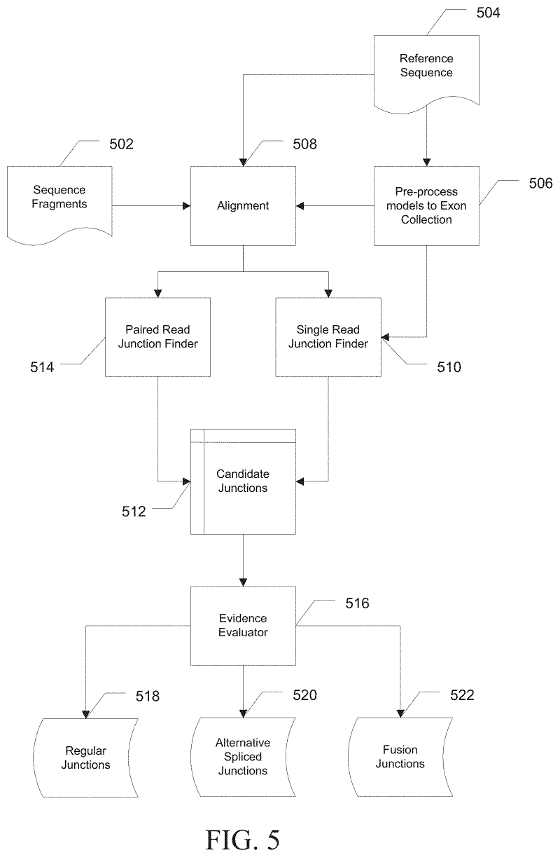

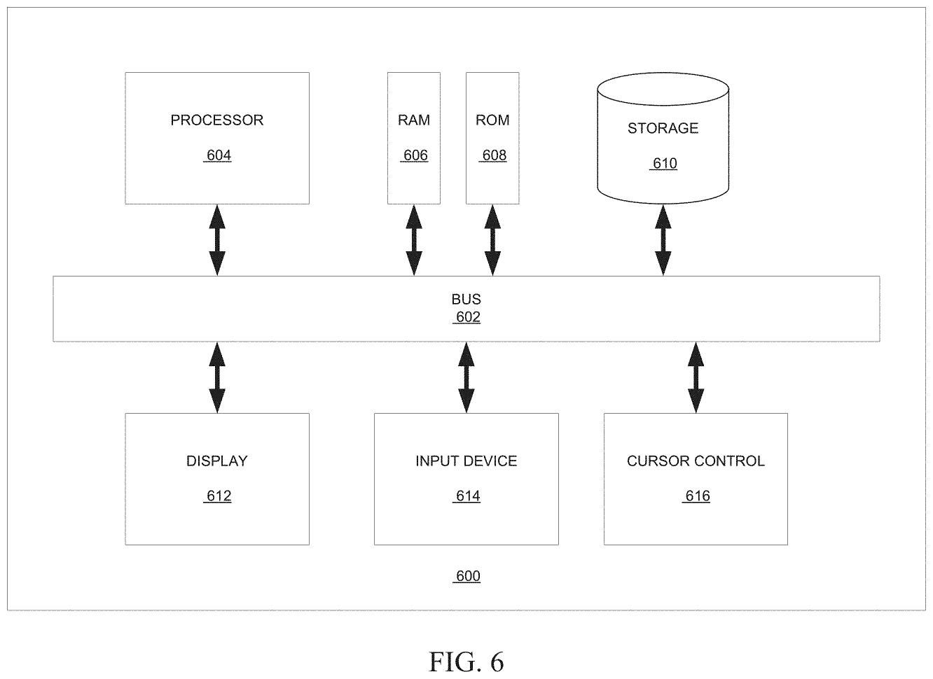

FIG. 5 illustrates an exemplary method for identifying exon junctions. At 502, the processor can obtain fragment three. The fragment reads can be produced from a fragment library, a mate pair library, or any combination thereof. The library can be derived from RNA, such as a whole transcript home or isolated messenger RNA.

At 504, the processor can obtain a reference sequence, and process the reference sequence to produce an exon collection, as shown at 506. At 508, the processor can align the sequence reads to the reference sequence. In particular embodiments, the processor can identify sequence reads that map to exons within the exon collection.

At 510, the processor can perform a single read junction finding method on the sequence reads. In particular embodiments, certain sequence reads can be excluded from the single read junction finder method. For example, if a read has already been completely mapped to a portion of the reference sequence, it can be assumed that it falls completely within an exon, within an intron, or spans an adjacent exon and intron. Thus, the sequence read does not span a junction, so it can be excluded from consideration by the single read junction finder method. Similarly, reads that have been mapped to a junction by a prior step are not of interest, because it is assumed that such evidence has already been registered. Briefly then, a read is admissible only if it is unmapped or has been only partly mapped.

In various embodiments, the single rejection finder method can attempt to map a first portion of the sequence read to a first exon and map a second portion of the sequence read to a second exon. Provided the sum of the length of the first portion, the length of the second portion, and a constant is substantially equal to the length of the sequence read, the sequence read can be identified as evidence of a junction between the first and second exons and can be added to a candidate junction list, as shown at 512.

At 514, the processor can perform a paired read junction finder method on the sequence reads. In particular embodiments, certain paired reads may be excluded from the pair read junction finder method. For example, if both the first read and the second read of a paired read map to the same exon, it can be assumed that the entire length of the mate-pair between the first and second read is within the exon. As such, the read does not span a junction, so it can be excluded from consideration by the paired read junction finder method. Similarly, reads that have been mapped to a junction by a prior step are not of interest, because it is assumed that such evidence has already been registered. Briefly then, a read is admissible only if it is unmapped or has been only partly mapped.

In various embodiments, the pair read junction finder method can map each read of the mate pair to exons within the reference sequence. A mate pair in which a first read maps to a first exon and a second read maps to a second exon can provide evidence of a junction between the first and second exon and can be added to a candidate junction list as shown at 512.

At 516, an evidence evaluator can evaluate the candidate junctions identified in the candidate junction table. The evidence evaluator can determine a likelihood that a candidate junction is not the result of an incorrect alignment and is the result of a transcript containing the identified exon exon junction. The evidence evaluator can consider an alignment quality, a number of candidates identifying the junction, or combinations thereof in evaluating a candidate junction.

In particular embodiments, the evidence evaluator can calculate a junction confident value (JCV) for each candidate junction. For example, the JCV can be calculated according to Equation 1.

$$\begin{matrix}Junction Confidence Value & \\ {JCV}_{{j}_{x - y}} = {\sum }_{i = 1}^{n} {PQV}_{i} - 10 {log}_{10} ( {EEM}_{{j}_{x - y}} ) . & Equation 1\end{matrix}$$

PQVᵢ is the phred-scale pairing quality value for the i'th unique paired read evidence for a candidate junction j_{x-y} and x and y are the junction exons and EEMⱼ_{_{x-y}} is the error expectation metric defined by Equation 2. For each unique single read evidence, the PQVᵢ can be set to 10. If there are multiple alignments for a given unique start point, the PQV of the first such alignment can be used.

$$\begin{matrix}Error expectation metric ( EEM ) & \\ {EEM}_{{j}_{x - y}} = \frac{{RC}_{x}}{\frac{{ℓ}_{x}}{{μ}_{T} + 3 \times {σ}_{T}}} \times \frac{{RC}_{y}}{\frac{{ℓ}_{y}}{{μ}_{T} + 3 \times {σ}_{T}}} . & Equation 2\end{matrix}$$

RC is the absolute proper mapped read count for the corresponding exon and l is the length of the exon; μ_{T} and σ_{T} are the mean and standard deviation of the insert size for the current experiment, T. Error expectation metric (EEM) can be used to quantify highly expressed junctions. This metric can be hard to calculate due to genome complexity and homology of exons. The estimation can consider the number of reads mapped to the exons, the length of, and a conservative insert range.

After the equation is calculated, a JCV that is larger than 100 can be set to 100 and if it is smaller than 0 it can be set to 0. A higher JCV can indicate increased confidence that the candidate junction is a real junction.

The processor can categorize identified junctions as regular junctions at 518, alternative splice junctions at 520, and fusion junctions at 522. Regular junctions can include exon junctions within a gene where the exons occur in the order that occurs in the gene. Alternative splice junctions can include exon junctions within the same gene in which the exons do not occur in the order that occurs in the genome. For example, a gene having a first, second, and third exons can produce an alternative spliced transcript in which the first and third exons are adjacent and the second exon is removed resulting in an alternative spliced junction between the first and third exons. A fusion junction can include an exon junction between exons from different genes.

FIG. 6 is a block diagram that illustrates a computer system 600, upon which embodiments of the present teachings can be implemented. Computer system 600 can include a bus 602 or other communication mechanism for communicating information, and a processor 604 coupled with bus 602 for processing information. Computer system 600 can also include a memory 606, which can be a random access memory (RAM) or other dynamic storage device, coupled to bus 602. Memory 606 can store data, such as sequence information, and instructions to be executed by processor 604. Memory 606 can also be used for storing temporary variables or other intermediate information during execution of instructions to be executed by processor 604. Computer system 600 can further include a read-only memory (ROM) 608 or other static storage device coupled to bus 602 for storing static information and instructions for processor 604. A storage device 610, such as a magnetic disk, an optical disk, a flash memory, or the like, can be provided and coupled to bus 602 for storing information and instructions.

Computer system 600 can be coupled by bus 602 to display 612, such as a cathode ray tube (CRT) or liquid crystal display (LCD), for displaying information to a computer user. An input device 614, such as a keyboard including alphanumeric and other keys, can be coupled to bus 602 for communicating information and commands to processor 604. Cursor control 616, such as a mouse, a trackball, a trackpad, or the like, can communicate direction information and command selections to processor 604, such as for controlling cursor movement on display 612. The input device can have at least two degrees of freedom in at least two axes that allows the device to specify positions in a plane. Other embodiments can include at least three degrees of freedom in at least three axes to allow the device to specify positions in a space. In additional embodiments, functions of input device 614 and cursor 616 can be provided by a single input devices such as a touch sensitive surface or touch screen.

Computer system 600 can perform the present teachings. Consistent with certain implementations of the present teachings, results are provided by computer system 600 in response processor 604 executing one or more sequences of one or more instructions contained in memory 606. Such instructions may be read into memory 606 from another computer-readable medium, such as storage device 610. Execution of the sequences of instructions contained in memory 606 can cause processor 604 to perform the processes described herein. Alternatively, hard-wired circuitry may be used in place of or in combination with software instructions to implement the present teachings. Thus, implementations of the present teachings are not limited to any specific combination of hardware circuitry and software.

The term “computer-readable medium” as used herein refers to any media that participates in providing instructions to processor 604 for execution. Such a medium may take many forms, including but not limited to, nonvolatile memory, volatile memory, and transmission media. Nonvolatile memory includes, for example, optical or magnetic disks, such as storage device 610. Volatile memory includes dynamic memory, such as memory 606. Transmission media includes coaxial cables, copper wire, and fiber optics, including the wires that comprise bus 602. Non-transitory computer readable medium can include nonvolatile media and volatile media.

Common forms of non-transitory computer readable media include, for example, floppy disk, flexible disk, hard disk, magnetic tape, or any other magnetic medium, a CD-ROM, any other optical medium, punch cards, paper tape, any other physical medium with patterns of holes, a RAM, a PROM, an EPROM, a FLASH-EPROM, and other memory chips or cartridge or any other tangible medium from which the computer can read.

Various forms of computer readable media may be involved in carrying one or more sequences of one or more instructions to processor 604 for execution. For example the instructions may initially be stored on the magnetic disk of a remote computer. The remote computer can load the instructions into its dynamic memory and send instructions over a network to computer system 600. A network interface coupled to bus 602 can receive the instructions and place the instructions on bus 602. Bus 602 can carry the instructions to memory 606, from which processor 604 can retrieve and execute the instructions. Instructions received by memory 606 may optionally be stored on storage device 610 either before or after execution by processor 604.

In accordance with various embodiments, instructions configured to be executed by processor to perform a method are stored on a computer readable medium. The computer readable medium can be a device that stores digital information. For example, a computer readable medium can include a compact disc read-only memory as is known in the art for storing software. The computer readable medium is accessed via processor suitable for executing instructions configured to be executed.

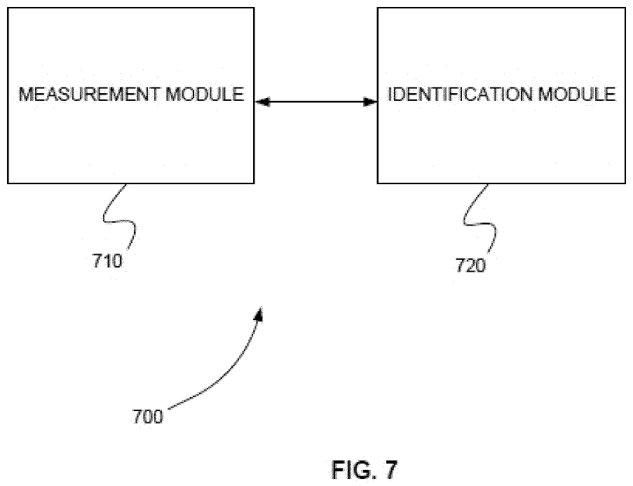

FIG. 7 is a schematic diagram of a system 700 of distinct software modules that performs a method for identifying an exon junction from a single read of a transcript, in accordance with various embodiments. System 700 can include measurement module 710 and identification module 720. Measurement module 710 can receive a read sequence from a nucleic acid sequencer that interrogates a transcript sample.

Identification module 720 can perform a number of steps. Identification module 720 can obtain the read sequence from the nucleic acid sequencer. Identification module 720 can obtain a first exon sequence and a second exon sequence. Identification module 720 can map the first exon sequence to a prefix of the read sequence. Identification module 720 can map the second exon sequence to a suffix of the read sequence. Identification module 720 can calculate a sum of a number of sequence elements of the first exon sequence that overlap the prefix of the read sequence, a number of sequence elements of the second exon sequence that overlap the suffix of the read sequence, and a constant. Finally, if the sum equals a length of the read sequence, identification module 720 can identify a junction in the read.

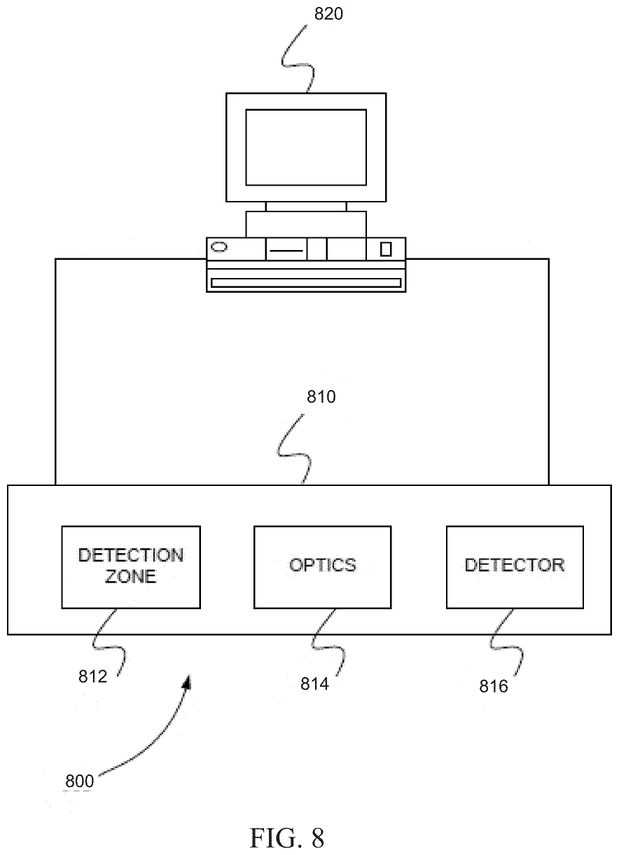

FIG. 8 is schematic diagram of a system 800 for identifying an exon junction from a single read of a transcript, in accordance with various embodiments. System 800 can include nucleic acid sequencer 810 and processor 820. Nucleic acid sequencer 810 can include, but is not limited to including, detection zone 812, optics 814, and detector 816. Nucleic acid sequencer 810 can be, but is not limited to, a next generation nucleic acid sequencing (NGS) system. Nucleic acid sequencer 810 can interrogate a transcript sample and can produce a read sequence from the transcript sample.

Processor 820 can be in communication with nucleic acid sequencer 810. Processor 820 can be, but is not limited to, a computer, microprocessor, or any device capable of sending and receiving control signals and data from nucleic acid sequencer 810 and processing data.

Processor 820 can perform a number of steps. Processor 820 can obtain the read sequence from nucleic acid sequencer 810. Processor 820 can obtains a first exon sequence and second exon sequence. The first exon sequence and second exon sequence can be obtained from a database, for example. The database can be a physical storage device with its own processor (not shown) that is connected to processor 820 across a network, or it can be a physical storage device connected directly to processor 820, for example. The first exon sequence and/or the second exon sequence can be a reverse sequence, for example.

Processor 820 can map the first exon sequence to a prefix of the read sequence. Processor 820 can map the second exon sequence to a suffix of the read sequence. Processor 820 can calculate a sum of the number of sequence elements of the first exon sequence that overlap the prefix of the read sequence, the number of sequence elements of the second exon sequence that overlap the suffix of the read sequence, and a constant. The constant can be 0 if the first exon sequence, the second exon sequence, and the read sequence are base-space sequences. The constant can be 0 if the first exon sequence, the second exon sequence, and the read sequence are monobase color-space sequences. The constant can be 1 if the first exon sequence, the second exon sequence, and the read sequence are dibase color-space sequences. If the sum equals a length of the read sequence, processor 820 can identify a junction in the read.

In various embodiments, processor 820 can map the first exon sequence to a prefix of the read sequence by at least a minimum number of sequence elements. The minimum number of sequence elements can be defined by a user, for example.

In a first aspect, a system for identifying an exon junction in a transcript sample can include a nucleic acid sequencer that interrogates the transcript sample and produces a read sequence from the transcript sample, and a processor in communication with the nucleic acid sequencer. The processor can be configured to obtain the read sequence from the nucleic acid sequencer, and obtain a first exon sequence and a second exon sequence. The processor can be further configured to map the first exon sequence to a prefix of the read sequence, and map the second exon sequence to a suffix of the read sequence. The processor can be further configured to calculate a sum of a number of sequence elements of the first exon sequence that overlap the prefix of the read sequence, a number of sequence elements of the second exon sequence that overlap the suffix of the read sequence, and a constant, and, if the sum equals a length of the read sequence, identify a junction in the transcript sample.

In an exemplary embodiment, the first exon sequence can be a reverse sequence.

In an exemplary embodiment, the second exon sequence can be a reverse sequence.

In an exemplary embodiment, the read sequence, the first exon sequence, the second exon sequence, and the read sequence can be base-space sequences and the constant can be 0.

In an exemplary embodiment, the read sequence, the first exon sequence, the second exon sequence, and the read sequence can be monobase color-space sequences and the constant can be 0.

In an exemplary embodiment, the read sequence, the first exon sequence, the second exon sequence, and the read sequence can be dibase color-space sequences and the constant can be 1.

In an exemplary embodiment, the processor can map the first exon sequence to a prefix of the read sequence by at least a minimum number of sequence elements. In a particular embodiment, the minimum number of sequence elements can be defined by a user.

In a second aspect, a system for identifying an exon junction in a transcript sample can include a processor. The processor can be configured to obtain a first read sequence, and obtain a first exon sequence and a second exon sequence. The processor can be further configured to map the first exon sequence to a prefix of the first read sequence, and map the second exon sequence to a suffix of the first read sequence. The processor can be further configured to calculate a sum of a number of sequence elements of the first exon sequence that overlap the prefix of the first read sequence, a number of sequence elements of the second exon sequence that overlap the suffix of the first read sequence, and a constant, and, if the sum equals a length of the read sequence, identify a junction in the transcript sample.

In an exemplary embodiment, the processor can be further configured to obtain a second read sequence, map the first exon sequence to a prefix of the second read sequence, and map the second exon sequence to a suffix of the second read sequence.

In an exemplary embodiment, the second read sequence can be a paired end read sequence.

In an exemplary embodiment, the processor can be further configured to calculate a confidence value for the junction. In a particular embodiment, the confidence value can depend on a number of unique read sequences corresponding to the junction.

In a third aspect, a method for identifying an exon junction can include interrogating a transcript sample and producing a plurality of read sequence using a nucleic acid sequencer, and obtaining a first read sequence of the plurality of read sequences from the nucleic acid sequencer using a processor. The method can further include obtaining a first exon sequence and a second exon sequence using the processor, mapping the first exon sequence to a prefix of the first read sequence using the processor, and mapping the second exon sequence to a suffix of the first read sequence using the processor. The method can further include calculating a sum of a number of sequence elements of the first exon sequence that overlap the prefix of the first read sequence, a number of sequence elements of the second exon sequence that overlap the suffix of the first read sequence, and a constant using the processor, and, if the sum equals a length of the read sequence, identifying a junction in the read using the processor.

In an exemplary embodiment, the first exon sequence can be a reverse sequence.

In an exemplary embodiment, the second exon sequence can be a reverse sequence.

In an exemplary embodiment, the read sequence, the first exon sequence, the second exon sequence, and the read sequence can be base-space sequences and the constant can be 0.

In an exemplary embodiment, the read sequence, the first exon sequence, the second exon sequence, and the read sequence can be monobase color-space sequences and the constant can be 0.

In an exemplary embodiment, the read sequence, the first exon sequence, the second exon sequence, and the read sequence can be dibase color-space sequences and the constant can be 1.

In an exemplary embodiment, the method can further include mapping the first exon sequence to a prefix of the read sequence by at least a minimum number of sequence elements. In a particular embodiment, the minimum number of sequence elements can be defined by a user.

In a fourth aspect, a method for identifying an exon junction in a transcript sample can include obtaining a first read sequence using a processor, and obtaining a first exon sequence and a second exon sequence using the processor. The method can further including mapping the first exon sequence to a prefix of the first read sequence using the processor, and mapping the second exon sequence to a suffix of the first read sequence using the processor. The method can further including calculating a sum of a number of sequence elements of the first exon sequence that overlap the prefix of the first read sequence, a number of sequence elements of the second exon sequence that overlap the suffix of the first read sequence, and a constant using the processor; and, if the sum equals a length of the read sequence, identifying a junction in the transcript sample using the processor.

In an exemplary embodiment, the method can further include obtaining a second read sequence, mapping the first exon sequence to a prefix of the second read sequence, and mapping the second exon sequence to a suffix of the second read sequence. In a particular embodiment, the second read sequence is a paired end read sequence.

In an exemplary embodiment, further comprising calculating a confidence value for the junction. In a particular embodiment, wherein the confidence value depends on a number of unique read sequences corresponding to the junction.

In a fifth aspect, a computer program product can include a non-transitory computer-readable storage medium whose contents include a program with instructions being executed on a processor so as to perform a method for identifying an exon junction.

The instructions can include instructions to obtain a first read sequence, and instructions to obtain a first exon sequence and a second exon sequence. The c instructions can further include instructions to map the first exon sequence to a prefix of the first read sequence, and instructions to map the second exon sequence to a suffix of the first read sequence.

Further, the instructions can include instructions to calculating a sum of a number of sequence elements of the first exon sequence that overlap the prefix of the first read sequence, a number of sequence elements of the second exon sequence that overlap the suffix of the first read sequence, and a constant, and instructions to identify a junction when the sum equals a length of the first read sequence.

In an exemplary embodiment, the first exon sequence can be a reverse sequence.

In an exemplary embodiment, the second exon sequence can be a reverse sequence.

In an exemplary embodiment, the read sequence, the first exon sequence, the second exon sequence, and the read sequence can be base-space sequences and the constant can be 0.

In an exemplary embodiment, the read sequence, the first exon sequence, the second exon sequence, and the read sequence can be monobase color-space sequences and the constant can be 0.

In an exemplary embodiment, the read sequence, the first exon sequence, the second exon sequence, and the read sequence can be dibase color-space sequences and the constant can be 1.

In an exemplary embodiment, the instructions can further include instructions to map the first exon sequence to a prefix of the read sequence by at least a minimum number of sequence elements. In a particular embodiment, the minimum number of sequence elements can be defined by a user.

In an exemplary embodiment, the instructions can further include instructions to obtain a second read sequence instructions to map the first exon sequence to a prefix of the second read sequence, and instructions to map the second exon sequence to a suffix of the second read sequence. In a particular embodiment, the second read sequence is a paired end read sequence.

In an exemplary embodiment, the instructions further comprise instructions to calculate a confidence value for the junction. In a particular embodiment, the confidence value depends on a number of unique read sequences corresponding to the junction.

While the principles of the present teachings have been described in connection with specific embodiments of control systems and sequencing platforms, it should be understood clearly that these descriptions are made only by way of example and are not intended to limit the scope of the present teachings or claims. What has been disclosed herein has been provided for the purposes of illustration and description. It is not intended to be exhaustive or to limit what is disclosed to the precise forms described. Many modifications and variations will be apparent to the practitioner skilled in the art. What is disclosed was chosen and described in order to best explain the principles and practical application of the disclosed embodiments of the art described, thereby enabling others skilled in the art to understand the various embodiments and various modifications that are suited to the particular use contemplated. It is intended that the scope of what is disclosed be defined by the following claims and their equivalents.

Further, in describing various embodiments, the specification may have presented a method and/or process as a particular sequence of steps. However, to the extent that the method or process does not rely on the particular order of steps set forth herein, the method or process should not be limited to the particular sequence of steps described. As one of ordinary skill in the art would appreciate, other sequences of steps may be possible. Therefore, the particular order of the steps set forth in the specification should not be construed as limitations on the claims. In addition, the claims directed to the method and/or process should not be limited to the performance of their steps in the order written, and one skilled in the art can readily appreciate that the sequences may be varied and still remain within the spirit and scope of the various embodiments.

The embodiments described herein, can be practiced with other computer system configurations including hand-held devices, microprocessor systems, microprocessor-based or programmable consumer electronics, minicomputers, mainframe computers and the like. The embodiments can also be practiced in distributing computing environments where tasks are performed by remote processing devices that are linked through a network.

It should also be understood that the embodiments described herein can employ various computer-implemented operations involving data stored in computer systems. These operations are those requiring physical manipulation of physical quantities. Usually, though not necessarily, these quantities take the form of electrical or magnetic signals capable of being stored, transferred, combined, compared, and otherwise manipulated. Further, the manipulations performed are often referred to in terms, such as producing, identifying, determining, or comparing.

Any of the operations that form part of the embodiments described herein are useful machine operations. The embodiments, described herein, also relate to a device or an apparatus for performing these operations. The systems and methods described herein can be specially constructed for the required purposes or it may be a general purpose computer selectively activated or configured by a computer program stored in the computer. In particular, various general purpose machines may be used with computer programs written in accordance with the teachings herein, or it may be more convenient to construct a more specialized apparatus to perform the required operations.

Certain embodiments can also be embodied as computer readable code on a computer readable medium. The computer readable medium is any data storage device that can store data, which can thereafter be read by a computer system. Examples of the computer readable medium include hard drives, network attached storage (NAS), read-only memory, random-access memory, CD-ROMs, CD-Rs, CD-RWs, magnetic tapes, and other optical and non-optical data storage devices. The computer readable medium can also be distributed over a network coupled computer systems so that the computer readable code is stored and executed in a distributed fashion.

What is claimed is:

1. A system for identifying a fusion junction in a human transcriptome suspected of containing a gene fusion, the system comprising:
a nucleic acid sequencer configured to:
receive a plurality of nucleic acid fragments of a fragment library, the fragment library comprising nucleic acid fragments created from the human transcriptome,
provide reagents for sequencing the nucleic acid fragments, and
detect a plurality of signals during sequencing, the signals representative of a first sequence of at least one of the nucleic acid fragments;
a memory comprising a stored list of exon prefix sequences and a stored list of exon suffix sequences;
a processor in communication with the nucleic acid sequencer and the memory, the processor configured to:
obtain a first read sequence based on the detected plurality of signals from the nucleic acid sequencer, the first read sequence corresponding to the first sequence,
generate the stored list of exon prefix sequences by comparing exons of a human genome to the first read sequence and identifying the exons that have a prefix sequence mapping to a suffix sequence of the first read sequence,
generate the stored list of exon suffix sequences by comparing exons of the human genome to the first read sequence and identifying the exons that have a suffix sequence mapping to a prefix sequence of the first read sequence,
select a pair of exon sequences from the stored lists of exon prefix sequences and exon suffix sequences, a first exon sequence of the pair being one of the exon suffix sequences and a second exon sequence of the pair being one of the exon prefix sequences,
calculate a sum of a number of sequence elements of the first exon sequence that overlap the prefix of the first read sequence, a number of sequence elements of the second exon sequence that overlap the suffix of the first read sequence, and a constant, and
if the sum equals a length of the first read sequence, identify a fusion junction between exons associated with the first exon sequence and second exon sequence in the human transcriptome, and identify a presence of a gene fusion in the human transcriptome based on the identified fusion junction, and
if the sum does not equal a length of the first read sequence, repeat the selecting of a pair of exon sequences and calculating of the sum for a different pair of exon sequences from the stored lists of exon prefix sequences and exon suffix sequences.

2. The system of claim 1, wherein the first exon sequence is a reverse sequence.

3. The system of claim 1, wherein the second exon sequence is a reverse sequence.

4. The system of claim 1, wherein the processor is configured to:
generate the stored list of exon prefix sequences by identify the exons that have a prefix sequence mapping to a suffix sequence of the first read sequence by at least a minimum number of sequence elements; and
generate the stored list of exon suffix sequences by identifying the exons that have a suffix sequence mapping to a prefix sequence of the first read sequence by the minimum number of sequence elements.

5. The system of claim 1, wherein the exon prefix sequences and the exon suffix sequences of the stored lists comprise sequences of a length ranging from 10 to 40 bases.

6. A method for identifying a fusion junction in a human transcriptome suspected of containing a gene fusion, the method comprising:
preparing a fragment library from nucleic acids isolated from the human transcriptome;
providing a plurality of nucleic acid fragments of the fragment library to a sequencing instrument;
detecting a plurality of signals, at least some of which are representative of a first sequence of one of the nucleic acid fragments of the fragment library; and
using a processor to:
generate a first read sequence representative of a first nucleic acid fragment from the plurality of signals,
generate a list of exon prefix sequences by comparing exons of a human genome to the first read sequence and identifying the exons that have a prefix sequence mapping to a suffix sequence of the first read sequence,
generate a list of exon suffix sequences by comparing exons of the human genome to the first read sequence and identifying the exons that have a suffix sequence mapping to a prefix sequence of the first read sequence,
select a pair of exon sequences from the lists of exon prefix sequences and exon suffix sequences, a first exon sequence of the pair being one of the exon suffix sequences and a second exon sequence of the pair being one of the exon prefix sequences,
calculate a sum of a number of sequence elements of the first exon sequence that overlap the prefix of the first read sequence, a number of sequence elements of the second exon sequence that overlap the suffix of the first read sequence, and a constant,
if the sum equals a length of the first read sequence, identify a fusion junction between exons associated with the first exon sequence and second exon sequence in the human transcriptome using the processor, and identify a presence of a gene fusion in the human transcriptome based on the identified fusion junction, and
if the sum does not equal a length of the first read sequence, repeat the selecting of a pair of exon sequences and calculating of the sum for a different pair of exon sequences from the lists of exon prefix sequences and exon suffix sequences.

7. The method of claim 6, further comprising using the processor to:
generate a second read sequence representative of a second nucleic acid fragment from the plurality of signals;
generate a second list of exon prefix sequences by comparing exons of the human genome to the second read sequence and identifying the exons that have a prefix sequence mapping to a suffix sequence of the second read sequence;
generate a second list of exon suffix sequences by comparing exons of the human genome to the second read sequence and identifying the exons that have a suffix sequence mapping to a prefix sequence of the second read sequence;
select a second pair of exon sequences from the second list of exon prefix sequences and the second list of exon suffix sequences, a first exon sequence of the second pair being one of the exon suffix sequences of the second list of exon suffix sequences and a second exon sequence of the second pair being one of the exon prefix sequences of the second list of exon prefix sequences;
calculate a sum for the second pair of a number of sequence elements of the first exon sequence of the second pair that overlap the prefix of the second read sequence, a number of sequence elements of the second exon sequence of the second pair that overlap the suffix of the second read sequence, and a constant;
if the sum for the second pair equals a length of the second read sequence, identify a second fusion junction between exons associated with the first exon sequence and second exon sequence in the human transcriptome using the processor, and identify a presence of a second gene fusion in the human transcriptome based on the identified second fusion junction; and
if the sum for the second pair does not equal a length of the second read sequence, repeat the selecting of a pair of exon sequences and calculating of the sum for a different pair of exon sequences from the second list of exon prefix sequences and the second list of exon suffix sequences.

8. The method of claim 7, wherein the second read sequence is a paired end read sequence.

9. The method of claim 6, further comprising using the processor to calculate a confidence value for the fusion junction.

10. The method of claim 9, wherein the confidence value depends on a number of unique read sequences corresponding to the fusion junction.

11. The method of claim 6, wherein the exon prefix sequences and the exon suffix sequences of the lists comprise sequences of a length ranging from 10 to 40 bases.

12. A computer program product, comprising a non-transitory computer-readable storage medium whose contents include a program with instructions being executed on a processor so as to perform a method for identifying a fusion junction in a human transcriptome suspected of containing a gene fusion, the instructions comprising:
instructions to receive a plurality of nucleic acid fragments of a fragment library, the fragment library comprising nucleic acid fragments created from the human transcriptome, into a sequencing instrument;
instructions to provide reagents for sequencing the nucleic acid fragments;
instructions to detect a plurality of signals during sequencing, at least a portion of the signals representative of a sequence of at least one of the nucleic acid fragments;
instructions to determine a first read sequence representative of the at least one nucleic acid fragment using the plurality of signals;
instructions to generate a list of exon prefix sequences by comparing exons of a human genome to the first read sequence and identifying the exons that have a prefix sequence mapping to a suffix sequence of the first read sequence,
instructions to generate a list of exon suffix sequences by comparing exons of the human genome to the first read sequence and identifying the exons that have a suffix sequence mapping to a prefix sequence of the first read sequence,
instructions to store the list of exon prefix sequences and the list of exon suffix sequences;
instructions to select a pair of exon sequences from the stored lists of exon prefix sequences and exon suffix sequences, a first exon sequence of the pair being one of the exon suffix sequences and a second exon sequence of the pair being one of the exon prefix sequences;
instructions to calculate a sum of a number of sequence elements of the first exon sequence that overlap the prefix of the first read sequence, a number of sequence elements of the second exon sequence that overlap the suffix of the first read sequence, and a constant;
instructions to identify a fusion junction between exons associated with the first exon sequence and second exon sequence when the sum equals a length of the first read sequence, and to identify a presence of a gene fusion in the human transcriptome based on the identified fusion junction; and
instructions to repeat the selecting of a pair of exon sequences and calculating of the sum for a different pair of exon sequences from the stored lists of exon prefix sequences and exon suffix sequences when the sum does not equal a length of the first read sequence.

13. The computer program product of claim 12, wherein the instructions further comprise:
instructions to generate a second read sequence representative of a second nucleic acid fragment from the plurality of signals;
instructions to generate a second list of exon prefix sequences by comparing exons of the human genome to the second read sequence and identifying the exons that have a prefix sequence mapping to a suffix sequence of the second read sequence;
instructions to generate a second list of exon suffix sequences by comparing exons of the human genome to the second read sequence and identifying the exons that have a suffix sequence mapping to a prefix sequence of the second read sequence;
instructions to store a second list of exon prefix sequences and a second list of exon suffix sequences;
instructions to select a second pair of exon sequences from the second lists of exon prefix sequences and exon suffix sequences, a first exon sequence of the second pair being one of the exon suffix sequences and a second exon sequence of the second pair being one of the exon prefix sequences;
instructions to calculate a sum for the second pair of a number of sequence elements of the first exon sequence of the second pair that overlap the prefix of the second read sequence, a number of sequence elements of the second exon sequence of the second pair that overlap the suffix of the second read sequence, and a constant;
instructions to identify a second fusion junction between exons associated with the first exon sequence and second exon sequence of the second pair when the sum for the second pair equals a length of the second read sequence, and
instructions to identify a presence of a second gene fusion in the human transcriptome based on the identified second fusion junction; and
instructions to repeat the selecting of a pair of exon sequences and calculating of the sum for a different pair of exon sequences from the stored second list of exon prefix sequences and second list of exon suffix sequences when the sum for the second pair does not equal a length of the second read sequence.

14. The computer program product of claim 13, wherein the second read sequence is a paired end read sequence.

15. The computer program product of claim 12, wherein the instructions further comprise instructions to calculate a confidence value for the fusion junction.

16. The system of claim 1, wherein the first read sequence has a length of 25-50 bases.

17. The system of claim 1, wherein the system is a next generation sequencing system.

18. The system of claim 1, wherein the fragment library comprises thousands of nucleic acid fragments.
